(19) United States
(12) Patent Application Publication
(10) Pub. No.: US 2025/0152815 A1
(43) Pub. Date: May 15, 2025

(54) PATIENT CARE SYSTEM FOR CRITICAL MEDICATIONS

(71) Applicant: ICU Medical, Inc., San Clemente, CA (US)

(72) Inventor: William Kenneth Day, Hoffman Estates, IL (US)

(21) Appl. No.: 19/026,018

(22) Filed: Jan. 16, 2025

Related U.S. Application Data
(63) Continuation of application No. 18/184,227, filed on Mar. 15, 2023.
Application No. 18/184,227 is a continuation of application No. 16/672,928, filed on Nov. 4, 2019, now Pat. No. 11,623,042.
Application No. 16/672,928 is a continuation of application No. 13/955,121, filed on Jul. 31, 2013, now Pat. No. 10,463,788.
(60) Provisional application No. 61/677,736, filed on Jul. 31, 2012.

(51) Int. Cl. A61M 5/172; A61M 5/142; G16H 20/17; G16H 40/40; G16H 70/40

(52) U.S. Cl. CPC A61M 5/172; G16H 20/17; G16H 40/40; A61M 2005/14208; A61M 2205/44; A61M 2205/50; A61M 2205/502; G16H 70/40

(57) ABSTRACT

A patient care system has a medical pump for delivering a medicine to a patient, and a processor in communication with the pump. The pump is configured to receive a first input on whether the medicine is a critical medicine, and a second input on a trigger condition that triggers a fail-operate mode for the critical medicine. The processor controls the medical pump to operate in the fail-operate mode, where the fail-operate mode continues delivery of the critical medicine when the trigger condition is triggered.

### BACKGROUND OF THE INVENTION

### Field of the Invention

Infusion pumps are used for intravenous delivery of medicines such as insulin, analgesics, sedatives, vasopressors, heparin and anti-arrhythmics to patients. Correct delivery of these medications is important for avoiding adverse events, particularly in critically ill patients. Smart infusion pumps, which include drug libraries and integrated decision support software in their medication delivery systems, have decreased errors in administration of medications by incorporating features such as hard and soft alarm limits, clinician messaging, and medication barcode input. Smart pumps are also able to utilize electronic medical records and inputs customizable for specific care units to improve safety for individual patients. Other infusion systems have incorporated features for a specific disease, such as algorithms to change the rates of insulin delivery based on a patient's glucose level, or to offer procedures specifically for advanced cardiac life support.

Yet, smart pumps are still subject to human programming errors and limited response times of busy clinicians. There remains a need to improve the ability for infusion pumps to provide safe delivery of medicines to patients, particularly in the case of critically ill patients where delivery of a medicine is life-sustaining.

### SUMMARY OF THE INVENTION

A patient care system has a medical pump for delivering a medicine to a patient, and a processor in communication with the pump. The pump is configured to receive a first input on whether the medicine is a critical medicine, and a second input on a trigger condition that triggers a fail-operate mode for the critical medicine. The processor controls the medical pump to operate in the fail-operate mode, where the fail-operate mode continues delivery of the critical medicine when the trigger condition is triggered.

### BRIEF DESCRIPTION OF THE DRAWINGS

Each of the aspects and embodiments of the invention described herein can be used alone or in combination with one another. The aspects and embodiments will now be described with reference to the attached drawings.

FIG. 1 shows an exemplary infusion pump in the art;

FIG. 2 is a schematic diagram of a typical medication delivery system;

FIG. 3 is an exemplary flowchart of general infusion pump operation known in the art;

FIG. 4 is a flowchart of an exemplary fail-operate infusion pump in one embodiment;

FIG. 5 shows an exemplary drug input screen for a fail-operate medical pump; and

FIG. 6 depicts an optional confirmation screen for an exemplary fail-operate mode.

### DETAILED DESCRIPTION OF THE PREFERRED EMBODIMENT

A method and system to allow infusion pumps to provide additional safety factors based on the type of drug being infused is disclosed. For certain patients and in certain care areas, the drug being infused is life-sustaining. In most failure or alarm modes of infusion pumps, the pump is designed to display an alarm and stop infusing when a particular condition is triggered in order to protect the patient from the failure. However, if the infusion is life-sustaining, this cessation in the delivery of the drug-referred to as a “fail-stop” condition in this disclosure—is likely to be more harmful than the potential risk of the alarm condition itself. In the present invention, a “fail-operate” mode is provided in which the pump continues infusing when the alarm condition is present. A clinician or pharmacist is enabled to identify critical drugs used in critical infusions, allowing the medications to sustain the patient while the alarm condition is present. The fail-operate mode, also referred to as a “keep infusing” mode in this disclosure, may be chosen for a certain care area in a hospital, and/or to be specific to a particular patient. This disclosure describes controls and methods of use to provide the clinician with the capabilities to customize particular alarm conditions to perform in a fail-operate mode rather than the normal fail-stop alarm condition response.

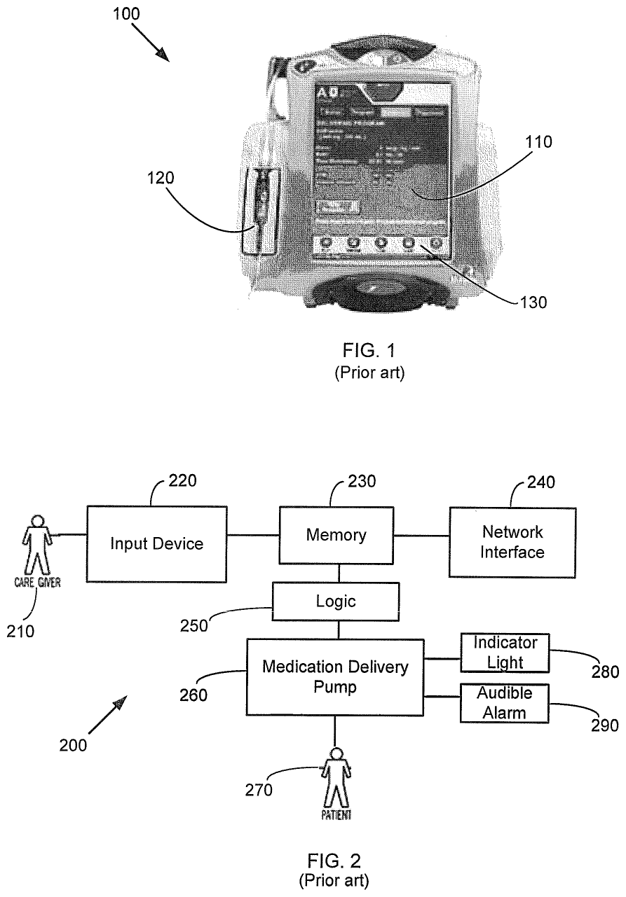

FIG. 1 shows an exemplary infusion pump 100, which includes a programming screen 110 and a cassette carriage 120 for loading an infusion administration set or line. The programming screen 110 is a touch screen in this embodiment, which allows a user to input or view various delivery data such as infusion settings and patient information. In addition, a menu bar 130 at the bottom of screen 110 includes buttons with which a professional caregiver may enter information or change a status regarding modes, settings, logs, locks, and alarms.

FIG. 2 illustrates a schematic diagram 200 of a typical medication delivery system, also referred to in the art as a medication management system. A caregiver 210 provides input through an input device 220, such as the programming screen 110 of FIG. 1, to enter delivery information and adjust settings as needed. The input device 220 stores information in a processor memory 230. A network interface 240, such as a Hospira MedNet™ platform, may also input information to memory 230. For example, network interface 240 may be in communication with a hospital information system or network, which may include a medication administration record system, pharmacy information system, or bar code-enabled point-of-care system. A processing logic system 250 utilizes information from processing memory 230 to control medication delivery pump 260, which infuses the desired fluids to the patient 270. The pump 260 may optionally include an indicator light 280 and/or an audible alarm 290 to provide sensory output indicating, for instance, when the infusion is in process or when an alarm condition occurs. The pump 260 may provide feedback to the logic system 250, such as with patient physiological parameters or pump failure parameters, so that the logic system 250 can monitor for the presence of any alarm thresholds.

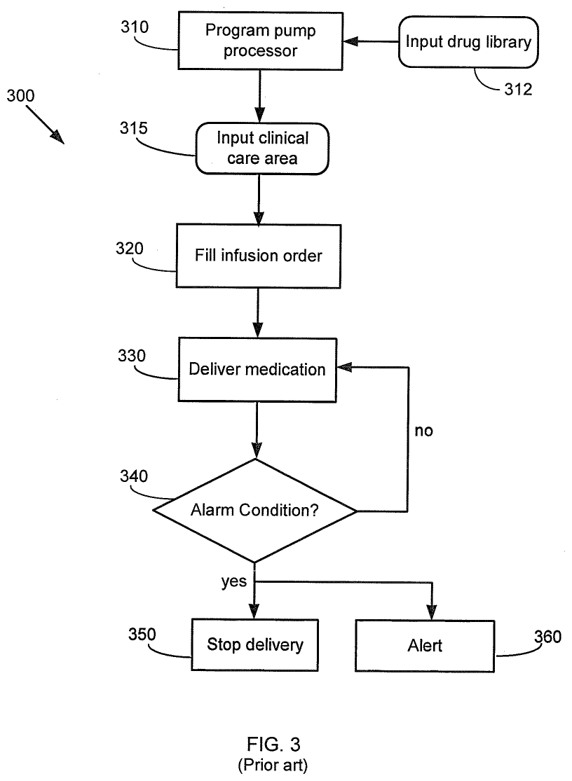

FIG. 3 is a flowchart 300 illustrating a general method of operation of smart infusion pumps known in the art. An infusion pump processor 310 receives drug library input 312 including customizable alarm limits via a central network. An infusion pump processor may also receive optional inputs such as basic patient data (e.g., weight, age) at a point of care of the patient. A medical caregiver may also input the clinical care areas (CCA) in which the pump is being used, in step 315. A caregiver then “fills the infusion order” by electronically downloading pump programming parameters via the network and/or manually programming the pump via the input device to deliver an infusion order for a patient in step 320, and the medication is delivered in step 330. In step 340, the processor checks whether an alarm condition is present. This alarm condition can be triggered when a failure of the pump occurs, such as occlusion in the delivery line, air-in-line, or low battery. Alarm threshold levels, such as for air in the infusion tubing, may be predetermined by the pump manufacturer, customized by a physician, biomed, pharmacist or other caregiver or they may be pre-set in the drug library. If the alarm condition is triggered, the pump stops delivery of the medicine in step 350. When failure occurs, an alert 360 may be sent. The alert may be in the form of an alarm sound or an electronic signal or message sent to a caregiver or physician to respond to the alarm. If no alarm condition is present, delivery of the drug continues.

Discontinuing delivery when a failure occurs, as in conventional smart pumps, is designed to protect the patient from potential adverse consequences of a pump “failure” or alarm condition. However, for critical drugs, discontinuing delivery may in fact harm a patient and even be life-threatening. Moreover, clinicians may not be immediately available to attend to a patient in such a case. Current infusion systems are designed to failstop, requiring critical, life-sustaining infusions to be carefully monitored by busy clinicians to prevent potentially noisy, annoying nuisance alarms from stopping the infusion until the alarm can be cleared.

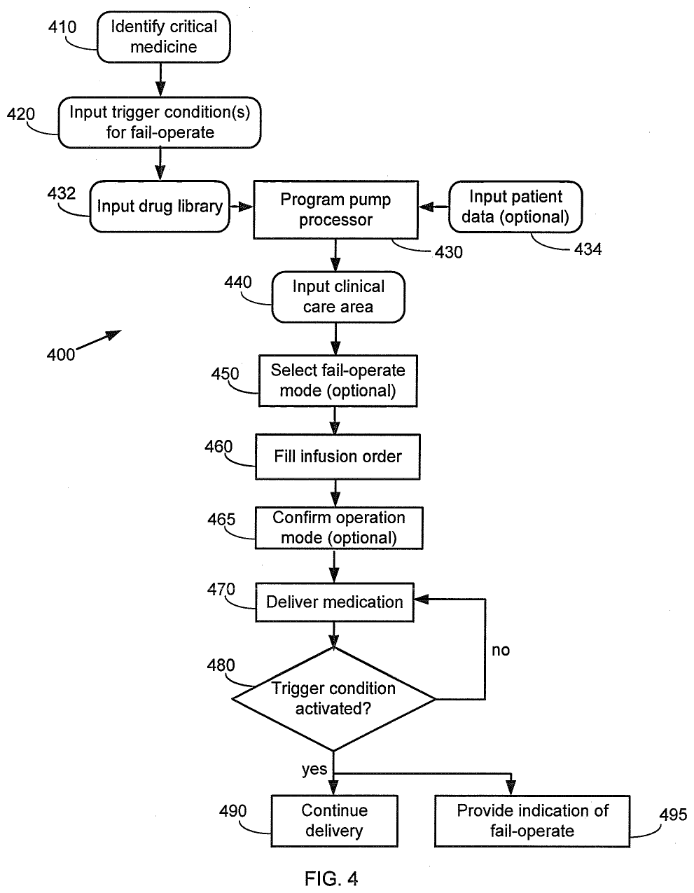

FIG. 4 is a flowchart 400 of one embodiment of the present disclosure in which a user is beneficially provided with an option to allow a drug to keep infusing in the presence of an alarm condition. In step 410, a medical professional such as a pharmacist or physician may identify or designate a drug in a drug library as a critical medicine. This input may be performed by, for example, calling up the drug library, selecting the desired medication, and clicking a toggle box associated with the medication to indicate that the drug is a critical medicine. Alternatively, the input may be performed by a medical professional or a caregiver through a touch screen on the pump, through keyboard or touch entry. One or more drugs may be specified in this input step. In some embodiments, a possible list of critical drugs may be provided by the drug library database, and the caregiver or medical professional may select or confirm particular drugs from this list. The critical drug input is then stored in memory as part of the drug library information. Critical drugs may include, for example, anticoagulants (e.g., heparin), saline or other fluids for hydration, nutrient solutions, and antibiotics. This selection of particular drugs in step 410 beneficially enables, via later steps described below, customized care for an individual patient based on their medical condition.

In step 420, trigger conditions at which a fail-operate mode are desired to be activated for the critical drug are input by a medical professional such as a pharmacist or physician. In some embodiments the trigger conditions may be standard alarm conditions, which may include an occlusion, air-in-line, or low battery. The conditions may be selected through, for example, a drop down menu in which a user selects to edit an alarm condition, and an editing screen then provides a toggle box to engage the fail-operate mode for that condition. In other embodiments the user may have the option to override all alarm conditions. In yet further embodiments, the user may have the option to adjust threshold values for the alarm, such as for an amount of air allowed to pass in the infusion line.

In step 430, drug library information 432, including the critical medicine information 410 and trigger condition information 420, is programmed into the processor. Optionally, patient data 434 may also be input, including the patient's medical history, recent medical treatments, and medical conditions such as monitored or tested physiological parameters related to critical drug infusions. The inputs 432 and 434, as well as subsequent inputs of flowchart 400, are stored in the memory of the processor, which is in communication with the infusion pump.

In step 440, a medical caregiver may also input one or more specific clinical care areas (CCA) in which the selected drug(s) may be used as a critical infusion. The ability to select or customize at the drug library particular CCA's in which the fail-operate mode is allowed for the critical drug enables further safety and customization for patients. For example, a certain antibiotic may be life-sustaining in an emergency room setting, while in a pediatric area it would be allowable to stop administration of the antibiotic without creating a life-threatening situation to the patient.

In some embodiments, the identification of a particular drug as a critical medicine in step 410 will automatically activate the fail-operate mode, when that drug is delivered. In other embodiments, a professional caregiver—such as the pharmacist or physician—may be allowed to choose whether to engage the fail-operate mode, as shown in optional step 450. This option may be useful, for example, to allow a physician to choose the fail-operate mode depending on the patient's condition. In some embodiments, the fail-stop mode may remain the default mode, even when settings for a fail-operate mode have been entered (e.g., in steps 410, 420, 440), unless activation of the fail-operate mode is confirmed before delivery. The options of customizing the fail-operate mode for particular critical drugs, for particular CCA's, and for particular patients, beneficially enables clinically targeted selection of the fail-operate mode for critical infusions. Additionally, enabling the physician to choose whether to engage the fail-operate mode provides even further safety to the patient compared to current infusion systems which operate only by stopping infusion when an alarm is triggered.

Still referring to FIG. 4, after the fail-operate conditions have been established, the infusion pump is ready to deliver medication. Upon receiving an order, a nurse programs the infuser to fill the order in step 460. During programming, the nurse may be informed by the delivery pump that the drug is a critical drug, and may optionally be presented with a confirmation screen in step 465 to confirm whether the infusion pump will use the fail-operate mode. The confirmation process may include a listing of the alarm conditions that will trigger the fail-operate mode. After filling the order, the medication is delivered in step 470. If an alarm condition is detected during infusion, the processor's logic system will determine in step 480 whether the alarm condition is one of the selected fail-operate trigger conditions. If the trigger condition is present, the infuser will provide an indication in step 495 that the trigger condition has been activated, but will continue to infuse if possible in step 490. Infusion typically will continue at the same rate as its existing rate. If the pump is unable to continue delivery, for example because of a fully blocked infusion line, it may be programmed to return to the fail-stop mode. In another embodiment, the infusion may continue but at a reduced non-zero rate.

Various indicators may be utilized in step 495 to alert personnel when the fail-operate mode is active. In some embodiments, triggering of the fail-operate mode may initiate an audible alarm, such as a single tone or a melody, where the sound for the fail-operate mode is different from that of the normal fail-stop mode. This differentiation in alarm sound beneficially alerts a caregiver that a fail-operate alarm condition exists, and that delivery of the critical medication is continuing. In other embodiments, triggering of the fail-operate mode may activate an alert light on the pump, display a text message on the pump's screen monitor, or send a signal or message notification to a clinician or a mobile communication device carried by the clinician. One or more of the indicators described herein may be used simultaneously. After the fail-operate mode has been engaged, the caregiver may locally clear the cause of the alarm condition and continue the infusion.

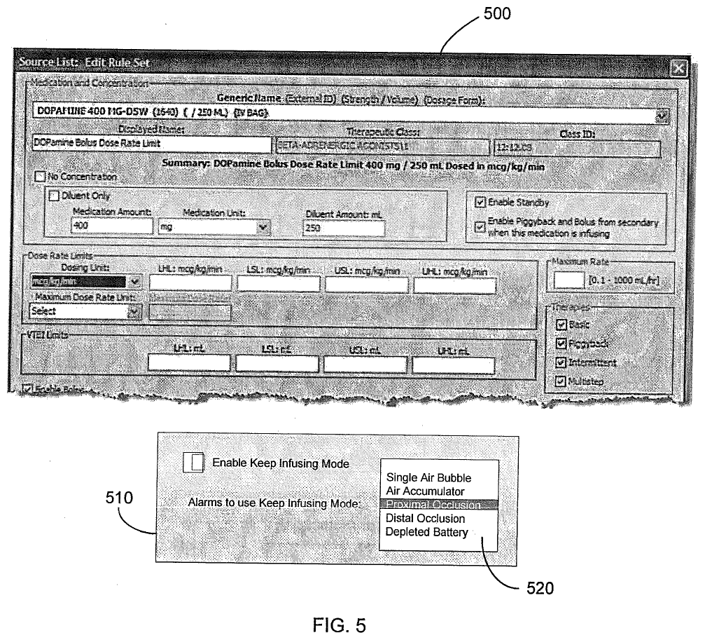

FIG. 5 shows an exemplary drug library editing screen 500, such as might appear on a display screen associated with a network interface such as a processor equipped with Hospira MedNet™ software would occur in steps 410 and 420 of FIG. 4. Editing screen 500 includes standard customizable fields, such as medication amount and dosing rate, for the drug dopamine which is used in this example. In the embodiment of FIG. 5, editing screen 500 includes a fail-operate input field 510 which allows the user to select “Enable Keep Infusing Mode” to engage the fail-operate condition. Selection in this embodiment is performed through marking a toggle box. In other embodiments, a user may be presented with the choice of “Keep Infusing” and “Stop Infusing” modes, and asked to highlight their desired choice, where the default may be shown as “Stop Infusing.” The user can further select one or more trigger alarm conditions in the subfield 520, which includes single air bubble, air accumulator, proximal occlusion, distal occlusion, and depleted battery in this example. This subfield 520 may be used, for example, in relation to step 420 of FIG. 4, and may further include a subsequent screen to input values associated with a particular condition. For example, after selecting “air accumulator,” the system may prompt the user to alter an allowable value of air accumulated, or to utilize a standard pre-set value.

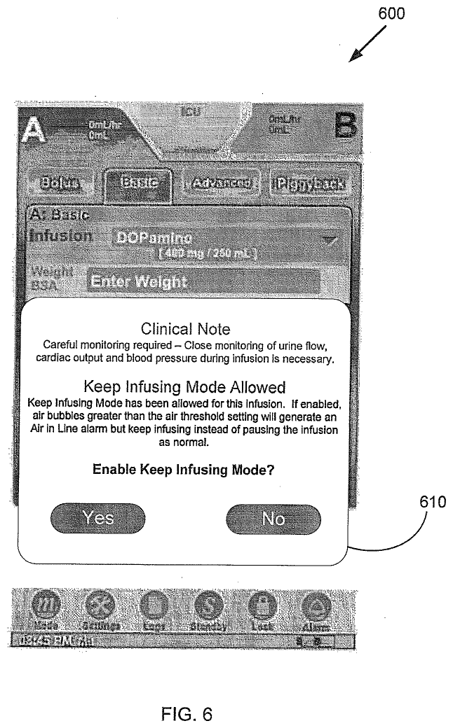

FIG. 6 shows an exemplary confirmation screen 600 at the pump 100, as would be used in association with steps 440 and 450 of FIG. 4. When a nurse fills an order for a drug, which again is shown as dopamine in this example, a pop-up screen 610 or other visual or audio signal notifies the nurse that the “Keep Infusing Mode” has been allowed. The nurse can then confirm whether to continue with the “Keep Infusing Mode” by selecting a yes or no response, and the system may be programmed to not proceed until the response is entered. The pop-up screen 610 in this example also lists information about the conditions that have been set for the Keep Infusing Mode. In some embodiments, the pop-up screen may be accompanied by an audible tone to alert the caregiver to verify the information on the display, to increase awareness to the caregiver that the pump has been programmed to operate in a different mode.

Various alarm or failure conditions are possible for triggering a fail-operate condition. For example, “fail-operate” could be used to continue delivery of a critical medication when air is present in the delivery line. In some pumps, there are two types of “air-in-line” alarms—single bubble and accumulated. Single bubble alarms are typically restricted in the drug library or in the pump code to approximately 50-500 microliters of air before alarming. Accumulated air alarms are typically set on the order of 1000 micro liters, as many patients are able to withstand more than a single bubble before harm occurs. In one embodiment, a fail-operate mode may be allowed to override the single bubble alarm and enable critical drug infusion to continue if the patient. Allowing a single bubble to pass may be more beneficial to the patient than halting delivery of a life-sustaining medicine, as the patient is still protected by the accumulated air alarm.

In another embodiment, an occlusion condition may operate in the fail-operate mode. Occlusions may occur when, for example, there is a kink in the infusion tubing, or when the tubing has been improperly loaded. The fail-operate mode allows the critical medication to continue being delivered, even at a partial rate due to a blockage in the line, to sustain the patient rather than stopping delivery until the line is fixed.

In another embodiment, the designation of a medication as a critical medication in a drug library can be optional and the medication can simply be flagged in the drug library downloaded to the pump or the memory resident in the pump as designated for delivery in a fail-operate mode. This embodiment provides a patient care system that includes a medical pump for delivering a medicine to a patient and a processor in communication with the medical pump. The processor has a memory and logic adapted to: 1) receive a first input on whether the medicine is allowed to be delivered in a fail-operate mode; and 2) receive a second input on a trigger condition that triggers the fail-operate mode. The fail-operate mode provides for continuation of the delivery of the medicine when the trigger condition is triggered. The first input and the second input are stored in the memory. The pump is controlled to operate in the fail-operate mode for the flagged medicine when the trigger condition is triggered. Manufacturers, institutions or users may flag only some drugs, flag entire drug libraries, all drugs in certain CCAs, or all drugs in certain pumps to be delivered in a fail-operate mode.

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those of ordinary skill in the art, without departing from the scope of the present invention. Furthermore, those of ordinary skill in the art will appreciate that the foregoing description is by way of example only, and is not intended to limit the invention. Thus, it is intended that the present subject matter covers such modifications and variations.

What is claimed is:

1. A patient care system comprising:
a medical pump for delivering a medicine to a patient; and
a processor in communication with the medical pump, the processor comprising a memory and a logic adapted to:
receive a first input on whether the medicine is a critical medicine;
receive a second input on a trigger condition that triggers a fail-operate mode for the critical medicine, wherein the fail-operate mode continues delivery of the critical medicine when the trigger condition is triggered;
store the first input and the second input in the memory; and
control the pump to operate in the fail-operate mode when triggered.

2. The system of claim 1 further comprising a fail-stop mode, wherein the fail-stop mode stops delivery of the medicine when the trigger condition is triggered.

3. The system of claim 2 wherein the processor is adapted to enable a user to select between engaging the fail-stop mode or the fail-operate mode.

4. The system of claim 2 wherein the fail-stop mode is the default mode.

5. The system of claim 2 further comprising a first audible alarm for the fail-stop mode and a second audible alarm for the fail-operate mode, wherein the first audible alarm is different from the second audible alarm.

6. The system of claim 1 wherein the processor is programmed to request confirmation for engaging the fail-operate mode.

7. The system of claim 6 wherein the confirmation is presented by a pop-up screen on the medical pump.

8. The system of claim 1 wherein the first input is received from a drug library or a professional caregiver.

9. The system of claim 1 wherein the second input is received from one of a drug library and a professional caregiver.

10. The system of claim 1 wherein the trigger condition is configured to be specific to an individual patient or to a specific clinical care area.

11. The system of claim 1 wherein the trigger condition is selected from the group consisting of an occlusion, air-in-line, air accumulation, and low battery.

12. The system of claim 1 wherein the critical medicine is chosen from the group consisting of anticoagulants, hydration fluids, nutrient solutions, and antibiotics.

13. The system of claim 1 wherein the processor provides an indication of when the fail-operate mode is engaged, wherein the indication comprises one of the group consisting of:
a notification to a medical professional, a message displayed on the medical pump, an audible tone, and an indicator light.

14. A method for configuring delivery of a critical medicine, the method comprising:
providing a medical pump and a processor in communication with the medical pump, wherein the processor comprises a memory and a logic;
configuring the processor with an option for entering a first input on whether a medicine is a critical medicine;
configuring the processor with an option for entering a second input on a trigger condition that triggers a fail-operate mode, wherein the fail-operate mode continues delivery of the critical when the trigger condition is triggered;
storing the first input and the second input in the memory; and
controlling the pump to operate in the fail-operate mode when triggered.

15. The method of claim 11 further comprising the step of configuring the processor with a fail-stop mode, wherein the fail-stop mode stops delivery of the medicine when the trigger condition is triggered, and wherein the processor is adapted to enable a user to select between engaging the fail-stop mode or the fail-operate mode.

16. The method of claim 11 further comprising the step of providing a first audible alarm for the fail-stop mode and a second audible alarm for the fail-operate mode, wherein the first audible alarm is different from the second audible alarm.

17. The method of claim 11 wherein the trigger condition is configured to be specific to an individual patient or to a specific clinical care area.

18. The method of claim 11 wherein the trigger condition is selected from the group consisting of an occlusion, air-in-line, and low battery.

19. The method of claim 11 further comprising the step of providing an indication of when the fail-operate mode is engaged, wherein the indication comprises one of the group consisting of:
a notification to a medical professional, a message displayed on the medical pump, an audible tone, and an indicator light.

20. A patient care system comprising:
a medical pump for delivering a medicine to a patient; and
a processor in communication with the medical pump, the processor comprising a memory and a logic adapted to:
receive a first input on whether the medicine is allowed to be delivered in a fail-operate mode;
receive a second input on a trigger condition that triggers the fail-operate mode, wherein the fail-operate mode continues delivery of the medicine when the trigger condition is triggered;
store the first input and the second input in the memory; and
control the pump to operate in the fail-operate mode for the medicine when triggered.
